(19) United States
(12) Patent Application Publication
(10) Pub. No.: US 2023/0270570 A1
(43) Pub. Date: Aug. 31, 2023

(54) METHOD FOR CONTROLLING A PROSTHESIS OR ORTHESIS

(71) Applicant: Otto Bock Healthcare Products GmbH, Vienna (AT)

(72) Inventors: Thomas Hofmann, Vienna (AT); Dirk Seifert, Vienna (AT); Sven Zarling, Duderstadt (DE)

(21) Appl. No.: 18/016,829

(22) PCT Filed: Jul. 20, 2021
(86) PCT No.: PCT/EP2021/070283

§ 371 Date: Jan. 18, 2023

(30) Foreign Application Priority Data
Jul. 20, 2020 (DE) 10 2020 004 339.9

(51) Int. Cl. A61F 2/64; A61F 2/66

(52) U.S. Cl. CPC A61F 2/64; A61F 2/6607; A61F 2002/7635

(57) ABSTRACT

The invention relates to a method for controlling a prosthesis or orthesis of the lower extremity, which prosthesis or orthesis comprises an upper part (10) and a lower part (20) that is connected to the upper part (20) via a knee joint (1) and is mounted so as to be pivotable relative to the upper part (10) about a joint pin (15); wherein an adjustable resistance device (40) is situated between the upper part (10) and the lower part (20), by means of which resistance device a flexion resistance (Rf) in an early and middle standing phase is modified, during walking, on the basis of sensor data, following initial heel contact up to the middle standing phase; wherein, following the initial heel contact, the flexion resistance (Rf) is increased to a value at which further flexion is blocked or at least slowed; wherein the progression over time of the flexion resistance increase and/or the maximum achievable flexion angle (Af) is modified on the basis of the inclination of the ground or a height difference (ΔH) to be overcome.

The invention relates to a method for controlling a prosthesis or orthosis of a lower extremity, having an upper part and having a lower part which is connected to the upper part via a knee joint and is mounted so as to be pivotable relative to the upper part about a joint axis, wherein there is arranged between the upper part and the lower part an adjustable resistance device by means of which, during walking, a flexion resistance is changed on the basis of sensor data in an early and mid stance phase after initial heel contact up to the mid stance phase.

Artificial knee joints are used in prostheses and orthoses as well as in exoskeletons as a special case of orthoses. An artificial knee joint has an upper part and a lower part which are mounted so as to be pivotable relative to one another about a joint axis, the knee axis. In the simplest case, the knee joint is in the form of a single-axis knee joint, in which, for example, a pin or two bearing points arranged on a pivot axis form a single knee axis. Also known are artificial knee joints which do not form a fixed axis of rotation between the upper part and the lower part, but have either sliding or rolling surfaces or a plurality of link bars connected together in an articulated manner. In order to be able to influence the movement properties of the knee joints and obtain a movement behavior of the orthosis or prosthesis, or of the exoskeleton, that emulates natural gait behavior, there are provided between the upper part and the lower part resistance devices by means of which the resistance can be changed. Purely passive resistance devices are passive dampers, for example hydraulic dampers, pneumatic dampers, or dampers that change the movement resistance on the basis of magnetorheological effects. There are also active resistance devices, for example motors or other drives, which, via a corresponding connection, can be operated as generators or energy stores.

The knee joints, that is to say the prosthetic joints or orthotic knee joints, are fixed to the patient by attachment means. In the case of prosthetic knee joints, fixing generally takes place by means of a thigh socket, which receives a limb stump. Alternative types of fixing are likewise possible, for example by osseointegrated attachment means or by means of belts and other devices. In the case of orthoses and exoskeletons, the upper part and lower part are fixed directly to the thigh and the lower leg. The fastening devices provided for that purpose are, for example, belts, sleeves, cups or frame structures. Orthoses can also have foot parts for supporting a foot or shoe. The foot parts can be mounted in an articulated manner on the lower part.

DE 10 2013 011 080 A1 relates to a method for controlling an orthopedic joint device of a lower extremity, having an upper part and a lower part mounted in an articulated manner thereon, between which there is arranged a conversion device by means of which mechanical work from the relative movement during a pivoting of the upper part relative to the lower part is converted and stored at least in one energy store. The stored energy is re-supplied to the joint device in a time-delayed manner in order to assist the pivoting of the upper part and lower part in the course of the movement. Assistance of the relative movement takes place in a controlled manner. In addition to the conversion device there can be provided a separate damper which is in the form of a hydraulic damper or pneumatic damper and is adjustable, so that, by means of the damper device, the resistance can be influenced both in the flexion direction and in the extension direction during walking.

An artificial knee joint with the maximum extension that is constructionally achievable has a knee angle of 180°, a hyperextension, that is to say an angle of more than 180° on the posterior side, is generally not provided. Pivoting of the lower part posteriorly relative to the upper part is referred to as knee flexion, pivoting anteriorly or in a forward direction is referred to as extension. On initial contact, the foot is placed on the ground at the end of the swing phase at the beginning of the stance phase. When walking on a level surface, a so-called heel strike occurs in most cases, in which the foot is placed down heel first. If the artificial knee joint remains in an extended, straight position during the heel strike, this results in a direct transmission of force into the pelvis, which on the one hand is uncomfortable and on the other hand is in conflict with the natural gait pattern. Therefore, analogously to normal walking on a level surface, a so-called stance phase flexion is allowed in prostheses and orthoses, in which, after the heel strike, the knee joint flexes about the joint axis, optionally against a resistance force via the resistance device.

WO 2015/0101417 A1 discloses a prosthetic knee joint having an upper part and a lower part which are pivotably mounted on one another via a four-limbed joint system. The joint system is mounted on the lower part so as to be pivotable from a starting position against a spring force during a stance phase flexion, wherein the action line of the spring force is so aligned that a moment acting against an inflexion of the joint system is present.

In the control methods known hitherto, it is a problem that the flexion resistance in the stance phase is set permanently, so that it can be difficult in different gait situations to provide a comfortable gait behavior.

The object of the present invention is, therefore, to provide a method for controlling a prosthesis or orthosis of a lower extremity with which an improved gait behavior can be achieved in a simple manner for users with artificial knee joints.

According to the invention, said object is achieved by means of a method having the features of the main claim. Advantageous embodiments and developments of the invention are disclosed in the dependent claims, in the description and in the figures.

The method for controlling a prosthesis or orthosis of the lower extremity, having an upper part and having a lower part which is connected to the upper part via a knee joint and is mounted so as to be pivotable relative to the upper part about a joint axis, wherein there is arranged between the upper part and the lower part an adjustable resistance device by means of which, during walking, a flexion resistance is changed on the basis of sensor data in an early and mid stance phase after initial heel contact up to the mid stance phase, provides that, after the initial heel contact, the flexion resistance is increased to a value at which further flexion is blocked or at least slowed, wherein the temporal profile of the flexion resistance increase and/or the maximum achievable flexion angle is changed in dependence on the inclination of the surface or a height difference to be overcome. The height difference to be overcome is the height of a prosthetic foot or of a foot part or of a foot of a user of the artificial knee joint relative to a foot or a foot part on the contralateral side of the patient in the stance phase thereof, or the height difference relative to the level of the immediately preceding stance phase of the prosthetic foot, of the orthotic foot part or of the foot during walking. In addition to walking on a level surface, normal locomotion also includes walking on ramps, wherein the ramps are walked on both upward and downward, and walking on stairs, wherein in particular walking downstairs is distinguished from walking downward on ramps. It is provided to limit the maximum possible stance phase flexion after initial heel contact to an adjustable angle. Stance phase flexion after initial heel contact is allowed in order to avoid the direct transmission of force into the user's pelvis. The flexion damping is here increased in dependence on the knee flexion angle until a target angle is reached or at least not exceeded. If a target angle is reached in the stance phase, further flexion is blocked. Until then, in the case of an increasing flexion angle or knee flexion angle, which manifests itself as a reduction of the knee angle on the posterior side of the knee joint, is increased, so that, in the case of loads and flexion moments which do not lead to a further stance phase flexion up to the maximum permitted target angle, no blocking of the flexion movement takes place. The temporal profile of the flexion resistance increase and/or the maximum achievable flexion angle is here changed in dependence on the inclination of the surface or, in the case of a gait situation of climbing stairs, a height difference to be overcome. It is thus possible that the user is able to carry out a knee flexion during the stance phase without the risk of no longer reaching complete extension in the stance phase as a result of excessive stance phase flexion. Complex stump control or a conscious use of muscles that are still present is no longer necessary. This allows the user to walk in a very relaxed and effortless manner, in particular at low walking speeds.

A development of the invention provides that the maximum achievable flexion angle and/or the flexion angle at which the maximum flexion resistance is achieved is increased in the case of an increasingly steep surface. When walking downward, it is necessary to catch the body weight when stepping with the assisted side via a stance phase flexion. This is facilitated in that, in the case of an increasingly steep surface, that is to say if it is more steeply downhill, the maximum achievable flexion angle is increased, whereby a longer path for providing sufficient flexibility and a longer path for converting the movement energy into heat or electrical energy or into another energy store is provided. In addition, in the case of an increasingly steep surface, the maximum flexion resistance can be reduced in order to permit a further inflexion and an increase in the maximum achievable flexion angle. Such a larger flexion angle can occur, in addition to when walking on a steep surface, also in the case of so-called braking steps or when walking downstairs. If the maximum achievable flexion angle or the maximum flexion resistance is reached, a more or less developed plateau forms in the temporal knee angle profile, because a further knee flexion is prevented or made more difficult after the target angle has been reached or shortly before a flexion block is reached.

The flexion block or the increased flexion resistance can be maintained for a defined period of time in the plateau phase, and then the flexion resistance can be reduced. The subsequent reduction of the flexion resistance over a defined period of time, which can also be fixed in space by the reaching of an orientation for example of the upper part, of the lower part or of a connecting line between the upper part and the lower part, the flexion damping is lowered again, for example to an initial level of a stance phase damping. In order to generate as few tilting moments as possible in the upper part, the flexion damping is advantageously reduced progressively, wherein the period of time of the reduction can be dependent on the surface inclination or the orientation of the components relative to one another in space.

The flexion resistance can be reduced after the flexion block and after the maximum stance phase flexion angle has been reached or after the flexion resistance increase and after a stance phase flexion angle that is achievable at said resistance has been reached, if a measure of the maximum transverse force in the lower part exceeds a limit value dependent on the inclination of the surface and/or a leg cord exceeds a forward inclination dependent on the inclination of the surface and/or a measure of the hip moment initially exceeds and then falls below a limit value. A measure of the transverse force within the lower part, for example at a lower leg tube or a lower leg splint, is a possible indicator of the instantaneous gait phase. The measure can be the transverse force itself, but it can also be defined in dependence on the transverse force and be, for example, in relation to the known body weight or the body weight determined by means of a sensor on the orthosis or prosthesis. If the transverse force or the measure of the transverse force has exceeded a maximum value dependent on the inclination of the surface, a reduction of the flexion resistance can be initiated. The transverse force is a force component which acts perpendicular to the longitudinal extension of the lower part, in the case of an upright, extended leg the transverse force runs in the anterior-posterior direction in the sagittal plane. The transverse force value can be measured directly by means of a transverse force sensor, which in this variant of the invention is the only force sensor required to carry out the method.

Alternatively or in addition, the flexion resistance can be reduced again after a flexion block and after the maximum stance phase flexion angle has been reached or after the flexion resistance increase and after a flexion angle possible therewith has been reached, if a leg cord exceeds a forward inclination dependent on the inclination of the surface. The leg cord is regarded as being a connecting line between two defined points on the upper part and the lower part or on a component attached to the lower part. A preferred embodiment provides that there is used as the leg cord the connecting line between a hip rotation point and a foot point. In the case of the use of a prosthetic knee joint, the hip rotation point is determined by an orthopedic technician and defines the segment length of the thigh or upper part, which is defined as the distance between the joint axis or knee axis and the hip rotation point. The segment length of the lower part is defined by the distance between the knee axis and a foot point. There can be defined as the foot point, for example, the middle of the foot, the instantaneous center of a rolling movement, the end point of the perpendicular of the lower leg at the level of the sole of the foot part, of the prosthetic foot or on the ground, other points close to the ground are likewise suitable for defining a foot point. Because a foot part for supporting a natural foot that is still present is not necessary in the case of orthoses or exoskeletons, the distance from the ground to the joint axis can also be used. The position and/or the length of the leg cord provide reliable information about the orientation of the leg and the movement progression. The leg cord can be calculated or assessed by means of absolute angle sensors in conjunction with the known segment lengths, an absolute angle sensor and a knee angle sensor. If the leg cord exceeds a forward inclination relative to the surface, movement progression can be concluded therefrom, which allows the flexion block to be removed or the flexion resistance to be reduced further in order that a swing and an initiation of the swing phase can be achieved. The mid stance phase and the end of the mid stance phase are also detected with the exceeding of the forward inclination in dependence on the inclination of the surface. The inclination of the surface can be obtained, for example, from a determined angle in the ankle joint. The surface inclination can, however, also be determined in a different way.

Alternatively or in addition, the flexion resistance can be reduced after the flexion block and after the maximum stance phase flexion angle has been reached or after the flexion resistance increase and after a stance phase flexion angle achievable at said resistance has been reached, if a measure of the hip moment initially exceeds and then again falls below a limit value. The limit value is exceeded and fallen below in a single stance phase. The occurrence of a high hip moment in the stance phase is an indicator of the inclination of the surface, since when walking uphill on a steep ramp, for example, a high flexing hip moment is initially established, which reduces as the step is continued. The same is true for walking upstairs. If an extending hip moment is detected, this is an indicator for walking downhill on a ramp. The extending hip moment reduces as the step is continued in the stance phase, so that, in the case where a limit value is initially exceeded and subsequently fallen below, it is possible to derive the inclination of the surface in order to adjust the flexion resistance accordingly. The hip moment can be calculated by means of a knee moment and the known geometric relationships, by means of the orientation of the upper part in space, or from the orientation of the lower part in space and the knee angle. In addition to the hip moment as such, a parameter based thereon can also be used as a measure for the hip moment, for example a value or a characteristic number which is formed in dependence on the spatial position of the upper part and/or the body weight.

As an alternative to measuring the transverse forces directly, it is possible to determine the measure of the transverse force from a difference between transverse force components of an ankle moment and a knee moment. If the body weight of the user of the artificial knee joint is also taken into consideration for this purpose, the control and flexion resistances can be adjusted in a particularly individual manner.

The flexion resistance can be reduced again after it has been increased if a predefined knee flexion angle is exceeded, wherein the reduction is reduced to a level below a blocking level. The reduction can be reduced, for example, to an initial stance phase damping level, wherein the knee flexion angle can be exceeded in particular on steeper ramps, since the extent of the flexion damping increase is dependent on the inclination of the surface.

The reduction of the flexion resistance in dependence on the transverse force is relevant in particular in the case of braking steps, in particular in the case of braking steps on a level surface and when walking down ramps or stairs. The ramp-inclination-dependent leg cord angle or the forward inclination of the leg cord in dependence on the inclination of the ramp is crucial in particular in the case of shallow ramps or ramps with a moderate gradient in order to avoid excessive hip extension and allow inflection of the knee joint at the correct time.

The inclination of the surface can be calculated from a vertical and/or horizontal distance travelled in the preceding swing phase by the knee joint, in particular by a reference point in the vicinity of the sole of the foot, or from the ratio of a vertical and horizontal distance travelled in the preceding swing phase by the knee joint, but in particular by a reference point in the vicinity of the sole of the foot, as a displacement calculation criterion. For this purpose, sensor signals of an inertial measurement unit, for example, are evaluated and integrated over a defined period of time. This yields velocities and distances travelled, which can be used for calculating the surface inclination. The surface inclination is the ratio of the vertical distance travelled to the horizontal distance travelled. The distance travelled by a point in the vicinity of the sole of the foot, that is to say the distance travelled by a reference point, must here be calculated. For this purpose, the position of the lower part or lower leg part is determined at the beginning and at the end of the integration and, by means of geometric parameters and a simplified angle function, the distance travelled by the reference point or the foot relative to the inertial measurement unit or IMU is calculated.

The beginning of the stance phase to be controlled can be determined on the basis of an axial force impulse, a plantar flexion acceleration and/or an ankle moment. By means of a pure axial force sensor in a foot part or on the lower part, it can be determined when a foot is placed down. After a force-free phase or a phase without axial force, a spontaneous increase in an axial force component is detected and serves as a meaningful indicator of the beginning of the stance phase. Without a force sensor, a plantar flexion acceleration can be determined if the foot part is an articulated foot part or a prosthetic foot is mounted in an articulated manner on the lower part. Likewise, an ankle moment, which acts in the plantar flexion direction, can be determined and, after a moment-free phase effecting a plantar flexion, can be used as the starting point for the stance phase to be controlled.

A variant of the invention provides that the inclination of the surface is calculated from an evaluation of the flexion angle and of an absolute angle of an upper part or of a lower part or from the evaluation of two absolute angles of the upper part and lower part, as a kinematic criterion. The profile of the knee angle is acquired and determined together with an absolute angle of an upper part or of a lower part. Alternatively, the absolute angle of the upper part and lower part is used as the kinematic criterion, and the inclination of the surface is calculated therefrom. After the occurrence or the initial heel contact, different tangential gradients between the inclination of the surface and the knee angle are obtained in dependence on the surface inclination, so that, with knowledge of the particular tangential gradient, it is possible to derive the surface inclination. For that purpose, the knee angular velocity and the lower part angular velocity in space when walking can be determined. The quotient of the two angular velocities is calculated therefrom, wherein the inclination of the surface is determined on the basis of the changes of the quotient of the angular velocities.

Such a kinematic criterion or such a calculation, based on kinematic parameters, of the inclination of the surface can be used together with the displacement calculation criterion by means of the calculation of the vertical and/or horizontal distance travelled, wherein a weighted use of the respective criteria is possible. In addition to the equally weighted consideration of the calculated inclination from the movement of the lower leg and thigh and the calculated inclination on the basis of the displacement calculation data from the signals of an IMU, the kinematic criterion, for example, can be weighted less or used only in specific situations or gait situations as an additional determining parameter or error avoidance measure. For example, in critical situations when walking downstairs, the kinematic criterion can be used, in addition to the displacement calculation criterion, to avoid unintentional blocking or release of the knee joint.

There can be used as a further control parameter the position and/or orientation of a ground reaction force vector in relation to the prosthesis or orthosis. It is likewise possible that the detection of a roll-over of a foot part over an edge prevents a damping increase or further reduces the increased resistance, which is advantageous in particular when walking downstairs in the case of rolling of the assisted leg. The distances travelled for the displacement calculation criterion are calculated in particular from the IMU values of the lower part at the end of the preceding stance phase and at the beginning of the stance phase to be controlled, wherein the distance between the position of the IMU on the orthosis or prosthesis and the respective reference point and also the spatial positions at the end of the stance phase, that is to say at toe-off and on initial heel contact or heel strike, are known. Both the displacement calculation criterion and the kinematic criterion can be used individually for determining the surface inclination, wherein the selectivity of the sensor signals can also be a factor for the application of one criterion or the other criterion.

The starting point and the end point of the displacement integration can be determined by means of a state machine, wherein different sensor signals are monitored for different events, Such an event would be, for example, a loaded roll-over at the edge of a step, which can be recognized by the detection of an axial force with a simultaneous forward inclination at least of the lower part or of the leg cord. A loaded roll-over or a lifting of the orthosis or prosthesis as well as re-loading of the orthosis or prosthesis can likewise serve as a decisive feature for the start and end time of the displacement integration.

Exemplary embodiments of the invention will be discussed in more detail below on the basis of the appended figures. In the figures:

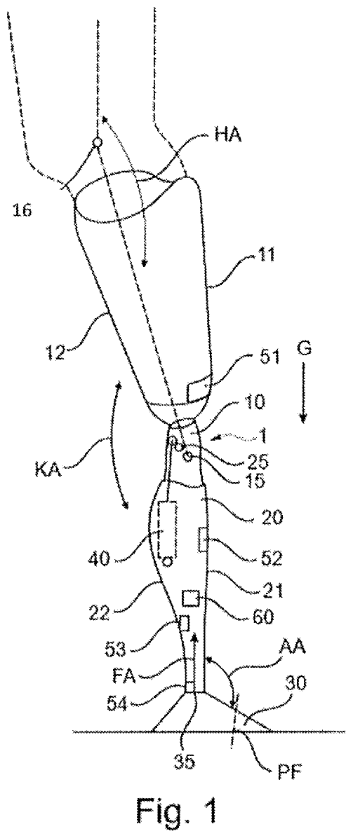

FIG. 1—shows a schematic illustration of a prosthetic leg;

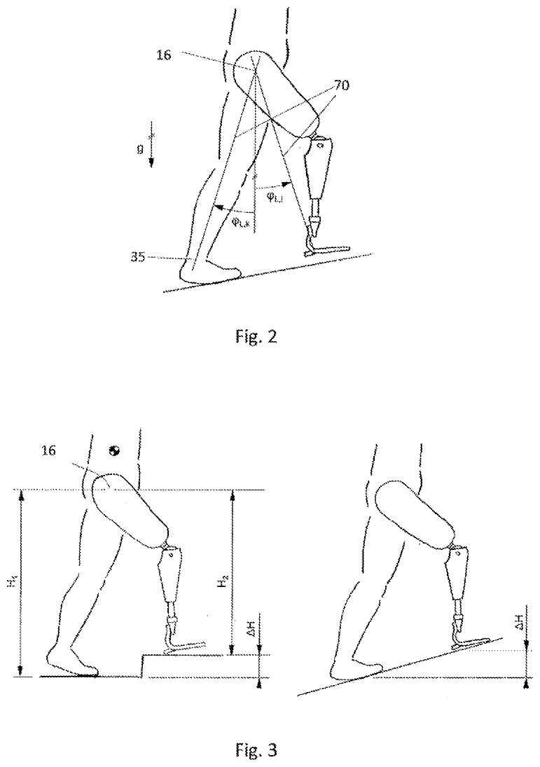

FIG. 2—shows an illustration of leg cords;

FIG. 3—shows a definition of a height difference when walking;

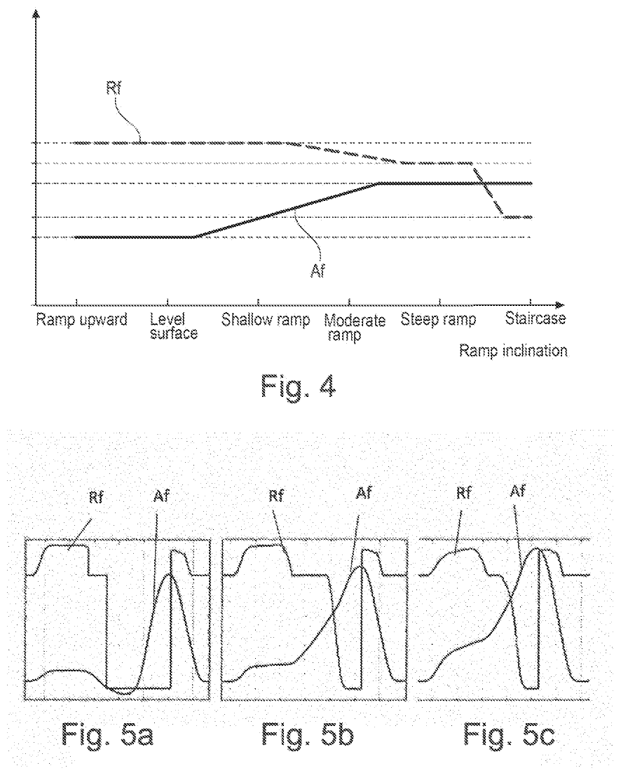

FIG. 4—shows an illustration of an inclination-dependent setting of the flexion angle and flexion resistance;

FIGS. 5a-5c—show different profiles of the flexion angle and flexion resistance at different surface inclinations;

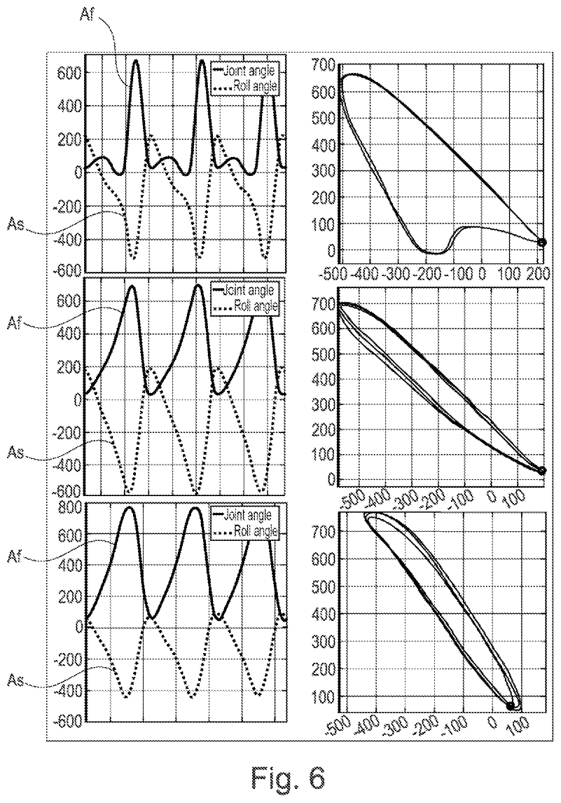

FIG. 6—shows illustrations of the flexion angle and roll angle at different surface inclinations:

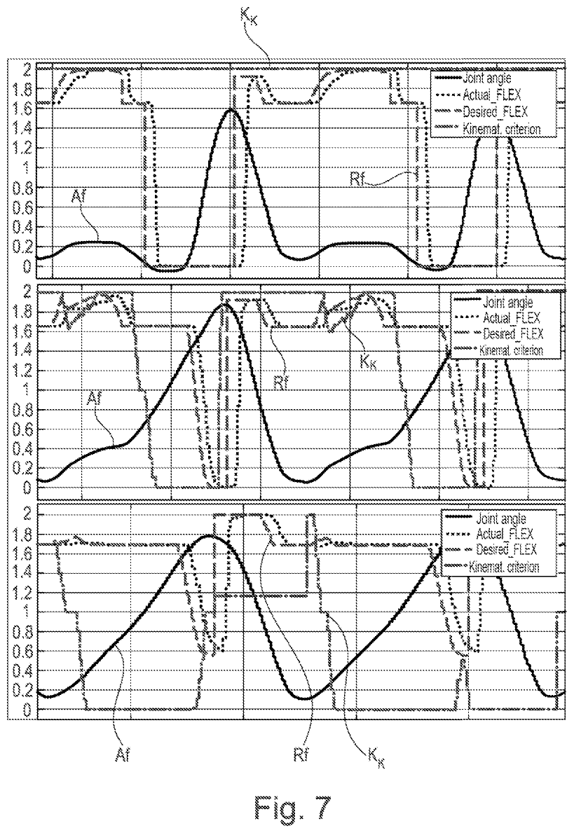

FIG. 7—shows illustrations of a kinematic criterion at different surface inclinations;

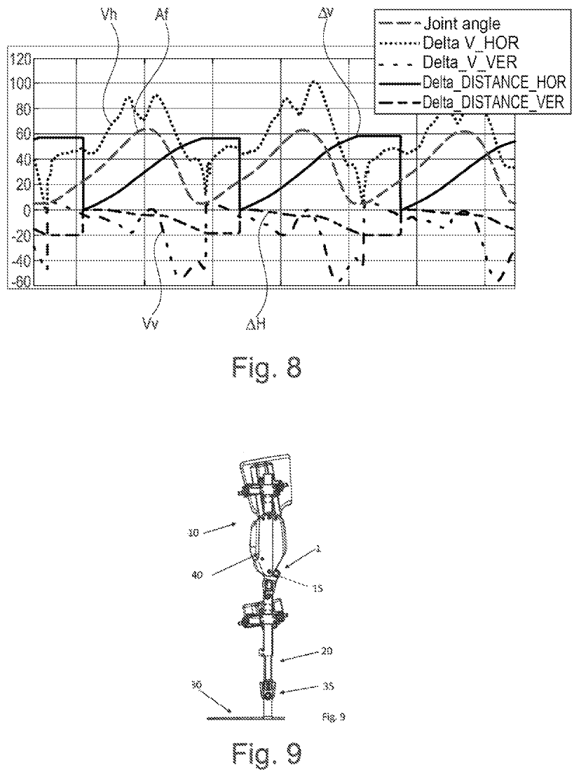

FIG. 8—shows an illustration of a geometric criterion when walking downward on a ramp, and

FIG. 9—shows an illustration of an orthosis.

FIG. 1 shows a schematic illustration of an artificial knee joint 1 in an application in a prosthetic leg. As an alternative to an application in a prosthetic leg, a correspondingly designed artificial knee joint 1 can also be used in an orthosis or an exoskeleton. Instead of replacing a natural joint, the artificial knee joint is then arranged medially and/or laterally on the natural joint. In the exemplary embodiment shown, the artificial knee joint 1 is in the form of a prosthetic knee joint having an upper part 10 with a side 11 which is anterior or situated in the walking direction or at the front, and a posterior side 12 which is located opposite the anterior side 11. A lower part 20 is arranged on the upper part 10 so as to be pivotable about a pivot axis 15. The lower part 20 also has an anterior side 21 or front side and a posterior side 22 or rear side. In the exemplary embodiment shown, the knee joint 1 is in the form of a monocentric knee joint, it is in principle also possible to control a polycentric knee joint in a corresponding manner. At the distal end of the lower part 20 there is arranged a foot part 30 which can be connected to the lower part either in the form of a rigid foot part 30 with a fixed foot joint or by a pivot axis 35, in order to make possible a movement sequence which emulates the natural movement sequence.

Between the posterior side 12 of the upper part 10 and the posterior side 22 of the lower part 20, the knee angle KA is measured. The knee angle KA can be measured directly by means of a knee angle sensor 25, which can be arranged in the region of the pivot axis 15. The knee angle sensor 25 can be coupled with a torque sensor or can have such a sensor, in order to detect a knee moment about the joint axis 15. On the upper part 10 there is arranged an inertial angle sensor or an IMU 51, which measures the spatial position of the upper part 10, for example in relation to a constant force direction, for example gravitational force G, which points vertically downward. An inertial angle sensor or an IMU 53 is likewise arranged on the lower part 20 in order to determine the spatial position of the lower part while the prosthetic leg is in use.

In addition to the inertial angle sensor 53, an acceleration sensor and/or transverse force sensor 53 can be arranged on the lower part 20 or on the foot part 30. By means of a force sensor or torque sensor 54 on the lower part 20 or on the foot part 30, an axial force FA acting on the lower part 20 or an ankle moment acting about the ankle joint axis 35 can be determined.

Between the upper part 10 and the lower part 20 there is arranged a resistance device 40 in order to influence a pivoting movement of the lower part 20 relative to the upper part 10. The resistance device 40 can be in the form of a passive damper, in the form of a drive, or in the form of a so-called semi-active actuator with which it is possible to store movement energy and purposively release it again at a later time in order to slow or assist movements. The resistance device 40 can be in the form of a linear or rotary resistance device. The resistance device 40 is connected to a control device 60, for example in a wired manner or via a wireless connection, which in turn is coupled with at least one of the sensors 25, 51, 52, 53, 54. The control device 60 electronically processes the signals transmitted by the sensors, using processors, computing units or computers. It has an electrical power supply and at least one memory unit in which programs and data are stored and in which a working memory for processing data is provided. After processing of the sensor data, an activation or deactivation command with which the resistance device 40 is activated or deactivated is outputted. By activation of an actuator in the resistance device 40 it is possible, for example, to open or close a valve or to generate a magnetic field, in order to change a damping behavior.

To the upper part 10 of the prosthetic knee joint 1 there is fastened a prosthesis socket, which serves to receive a thigh stump. The prosthetic leg is connected to the hip joint 16 by way of the thigh stump. On the anterior side of the upper part 10 a hip angle HA is measured, which is marked on the anterior side 11 between a vertical line through the hip joint 16 and the longitudinal extension of the upper part 10 and the connecting line between the hip joint 16 and the knee joint axis 15. If the thigh stump is lifted and the hip joint 16 is flexed, the hip angle HA decreases, for example when sitting down. Conversely, the hip angle HA increases in the case of an extension, for example when standing up or in the case of similar movement sequences.

During a gait cycle when waking on a level surface, the foot part 30 is placed down heel first, the first contact of the heel or of a heel part of the foot part 30 is called heel strike. A plantar flexion then takes place until the foot part 30 rests completely on the ground, the longitudinal extension of the lower part 10 is here generally behind the vertical, which runs through the ankle joint axis 35. When walking on a level surface, the center of mass is then displaced forward, the lower part 20 pivots forward, the ankle angle AA becomes smaller, and there is an increasing load on the forefoot. The around reaction force vector moves forward from the heel to the forefoot. At the end of the stance phase, a toe-off takes place, which is followed by the swing phase, in which the foot part 30, when walking on a level surface, is displaced behind the center of mass or the hip joint on the ipsilateral side, with a reduction of the knee angle KA, in order then, after a minimum knee angle KA has been reached, to be rotated forward in order then, with a knee joint 1 that is generally extended to the maximum, to achieve heel contact again. The force transmission point PF thus moves during the stance phase from the heel to the forefoot and is illustrated schematically in FIG. 1.

In FIG. 2, a definition of the leg cords 70 of an ipsilateral, assisted leg and of a contralateral, unassisted leg is given. The leg cord passes through the hip rotation point 16 and forms a line to the ankle joint 35, As can be seen in FIG. 2, the length of the leg cord and the orientation φ_{L} of the leg cords 70 changes during the movement, in particular also in the case of different gradients. The profile of the change of the length and/or orientation of the leg cords 70 can be used to assess and predict or determine height differences ΔH that are to be overcome. The respective control commands are then derived therefrom. The orientation of the ipsilateral leg cord φ_{Li} relative to the direction of gravity G as the vertical and of the contralateral leg cord φ_{Lk} is plotted in each case.

With reference to FIG. 3, the step height between the contralateral, unassisted leg and the ipsilateral foot part 30 of the assisted leg can be defined. For example, the distance H₁ from the ground to a distinctive point of the hip, for example the hip joint 16 or the trochanter major, at the level of the standing leg is defined, the distance H₂ is the distance between the ground and the hip joint 16 or the trochanter major on the leading side, which in the example shown is the assisted side. The height difference ΔH is then given by the difference between H₁ and H₂. A definition of the height difference ΔH for walking on a ramp applies correspondingly.

FIG. 4 shows an illustration of different settings of a flexion resistance Rf and a flexion angle Af. The flexion resistance Rf and the flexion angle Af are each set as maximum values. The maximum flexion damping or the maximum flexion resistance Rf remains almost constant when walking down a ramp, when walking on a level surface and on shallow ramps. Only when the downward inclination of the ramp increases is the maximum flexion resistance reduced, for example by 5%. For the situation of walking downstairs, the maximum flexion resistance Rf is then reduced to a substantially lower level, in particular to a level of the stance phase damping for walking on a level surface. The maximum flexion angle Af is likewise changed in dependence on the inclination of the surface. Walking up a ramp and walking on a level surface take place at the same maximum flexion angle Af as in the stance phase. The maximum achievable flexion angle Af is increased in dependence on the surface inclination up to a maximum value, which is set for walking on steep ramps and for walking downstairs. Such stance phase control adjusts the flexion resistance Rf during the early and mid stance phase in dependence on the inclination of the surface and thus also limits the maximum flexion angle within the stance phase. The maximum possible flexion angle Af is adjustable, wherein the limit value is effected by changing the flexion resistance Rf. In dependence on the surface inclination, a maximum target value is specified, at which the flexion resistance Rf has such a high value that no further knee flexion is possible. If the maximum flexion angle Af in dependence on the surface inclination is reached, a different knee angle plateau develops as the movement continues, that is to say when walking on a level surface, on a ramp or when climbing stairs, because further flexion is prevented. If it is recognized that an increased flexion angle is necessary, for example when walking on moderate or steep ramps or when climbing stairs, the flexion resistance Rf is reduced so that an increase of the flexion angle Af is possible also without a plateau, Thus, for example, for climbing stairs, the flexion resistance Rf is reduced to the stance phase damping level.

FIGS. 5a and 5b show temporal profiles of the flexion angle Af and flexion resistance Rf for different surface inclinations. The left-hand illustration in FIG. 5a shows walking on a level surface, the middle illustration in FIG. 5b shows the profile of the two parameters for walking on shallow ramps, the right-hand diagram in FIG. 5c shows the profile of the parameters when walking on ramps with a moderate inclination. In the illustration of FIG. 5a, a knee flexion is first effected after initial contact, so that the flexion angle Af increases. Together with the increase in the flexion resistance Rf, it is clear from the left-hand third of FIG. 5a that further flexion is suppressed, so that a plateau both of the flexion resistance Rf and of the flexion angle Af occurs. As the gait progression continues, after the roll-over, the flexion angle Af decreases. Then, from a specific limit value, the flexion resistance Rf is reduced in order to allow further flexion at the end of the stance phase, so that a sufficiently large flexion angle Af can be achieved in the swing phase.

FIG. 5b shows walking on a shallow ramp, Here too, the flexion resistance Rf is increased after the heel strike until further flexion and a further increase of the flexion angle Af is no longer possible. Unlike in FIG. 5a, the plateau phase in the first third of the diagram of FIG. 5b is not followed by an extension but by a roll-over with a flexed knee joint. Such a movement profile is typical for walking downward on a shallow ramp. The flexion resistance Rf is reduced after a defined period of time. The period of time can, for example, be such that, after the heel strike and at a normal walking speed, complete contact with the ground has been achieved by the foot part, Statistical data for the duration of a stance phase and thus also for the first lowering of the flexion resistance Rf can be used to initiate an inflexion. The flexion resistance Rf is lowered, so that a further inflexion and a further increase of the flexion angle Af can take place. Here too, the swing phase is allowed and the flexion resistance Rf is reduced to a minimum value.

FIG. 5c shows the profile of the parameters on a steeper surface, the plateau phase after the flexion resistance Rf has been increased is very short, the reduction of the flexion resistance Rf for the swing and initiation of the swing phase takes place as in FIG. 5b at the latest possible point in time, in order to obtain sufficient stability in particular when walking downhill. The flat portions in the profile of the knee angle Af are in each case accompanied by slowing of the initial knee flexion owing to the increased flexion resistance Rf. More ground clearance is thus generated during the swing of the contralateral, that is to say unassisted, side. Unnecessary compensating movements on the contralateral side can thus be avoided. The profiles of the flexion resistance Rf between the heel strike or initial heel contact and a renewed extension movement or in the case of a roll-over movement are of particular importance.

FIG. 6 shows three diagrams for different surface inclinations, the top illustration relates to walking on a level surface, the middle illustration relates to walking down a ramp, and the bottom illustration relates to walking downstairs. In the left-hand diagram in each case, the profiles of the flexion angle Af and the inclination angle or roll angle As of the lower part are shown. In the right-hand illustration in each case, the roll angle As is plotted as the X-component and the flexion angle Af is plotted as the Y-component. Heel contact is marked with a circle in each case, Starting from heel contact, the diagram passes through a closed, two-dimensional curve with in each case characteristic curve profiles. At selected points in time, for example in constant, discrete time portions, the tangential gradient of the curve profile is calculated. For this purpose, the ratio of the flexion angular velocity and the roll angular velocity is calculated continuously. The calculated ratio is optionally smoothed by means of a low-pass filter, wherein the filter is not switched on or initialized until shortly after the heel contact or heel strike, in order to avoid excessive signal interference owing to the initial heel contact. From the heel strike, via the starting stance phase flexion, the calculated and filtered value is assigned via an interpolation function with defined support points to a corresponding ramp inclination. It follows from the illustrations of FIG. 6 that, for each gait situation with different inclinations, different tangential gradients occur at the curve, so that the tangential gradient can be used to determined the surface inclination and to set a corresponding change of the flexion resistance in dependence on the surface inclination.

FIG. 7 shows three graphs for evaluating the kinematic criterion, that is to say for evaluating the flexion angle Af and the roll angle As of the lower part. The top graph illustrates walking on a level surface, the middle graph illustrates walking on a shallow ramp, the bottom illustration represents walking on a steep ramp. The curve profile of the flexion angle Af corresponds substantially to the profile of the flexion angle of FIGS. 5a to 5c. The profile of the flexion resistance Rf is shown in each case by the solid line, the dashed line is the time-delayed actual value. In the top illustration, the kinematic criterion Kk is set at the value 2 throughout, which corresponds to walking on a level surface. The value 1 corresponds to walking on a moderate ramp, the value 0 corresponds to a steep ramp or a staircase. It can be seen in the top illustration that the kinematic criterion Kk for walking on a level surface shows correct values throughout and provides correspondingly adjusted control of the flexion resistance Rf for the stance phase flexion with the pronounced plateau phase for the flexion angle Af. In the middle graph, it can be seen that the value for the kinematic criterion Kk falls to approximately 1.6, which corresponds to walking on a shallow ramp. As soon as the value of the flexion resistance Rf increases, a self-reinforcement process occurs, which raises the kinematic criterion Kk again in the direction of walking on a level surface. The minimum of the kinematic criterion Kk achieved after the heel strike occurs is therefore crucial for controlling the flexion resistance Rf. In the present case, this leads to an increase in the flexion resistance Rf up to a block at a significantly larger flexion angle Af than in the case of walking on a level surface.

In the bottom illustration of FIG. 7, it can be seen how the kinematic criterion Kk quickly falls to the value 0 when walking on very steep ramps, which is comparable to walking up stairs. An increase in the flexion resistance Rf is thus avoided, so that further flexion of the knee and an increase in the flexion angle Af are possible without a plateau phase. The surface that is being walked on or the gait situation that is recognized thus leads to significantly different changes of the flexion resistance Rf,

FIG. 8 illustrates the displacement calculation criterion, which is determined over a defined period of time on the basis of the sensor signals of the IMU. The accelerations at the knee joint 1 or the lower part 20 in the forward direction and upward direction are integrated. The upward direction acts against the direction of gravity, the forward direction is a forward direction in the sagittal plane from posterior to anterior. By means of a single integration over time, the velocities are obtained in each case, by means of a double integration over time of the values of the IMU, the distances travelled in the vertical direction and in the horizontal direction are obtained in each case. In FIG. 8, the horizontal distances travelled are denoted ΔV, the vertical distances travelled are denoted ΔH. The vertical velocities are described with Vv, the horizontal velocities with Vh. FIG. 8 shows walking on a downwardly inclined ramp. The general surface inclination is defined as the ratio of the horizontal distance travelled ΔV to the vertical distance travelled ΔH, wherein the distance travelled is in each case calculated in the vicinity of the sole of the foot. For this purpose, the position of the lower part 2 is determined at the beginning and at the end of the integration, and the distance travelled by the foot relative to the IMU can be calculated by means of the known geometric parameters such as length of the lower part, the position of the IMU on the lower part 20 or on the upper part 10, and the knee angle. Using the displacement calculation criterion, it is also possible to recognize the surface inclination with a comparatively good resolution. The value for the general surface inclination determined by means of the displacement criterion can then be used as a control parameter for adjusting the flexion resistance Rf.

FIG. 9 shows, in a schematic illustration, an exemplary embodiment of an orthosis having an upper part 10 and a lower part 20 mounted thereon so as to be pivotable about a pivot axis 15, with which the method can likewise be carried out. Between the upper part 10 and the lower part 20 there is formed an artificial knee joint 1, which in the exemplary embodiment shown is arranged laterally to a natural knee joint. In addition to an arrangement of the upper part 10 and lower part 20 on one side relative to a leg, it is also possible for two upper parts and lower parts to be arranged medially and laterally to a natural leg. The lower part 20 has at its distal end a foot part 30 which is mounted so as to be pivotable relative to the lower part 20 about an ankle joint axis 35. The foot part 30 has a foot plate on which a foot or shoe can be supported. Both on the lower part 20 and on the upper part 30 there are arranged fastening devices for fixing to the lower leg or the thigh. Devices for fixing the foot on the foot part 30 can also be arranged on the foot part 30. The fastening devices can be in the form of buckles, belts, clips or the like, in order to allow the orthosis to be releasably placed on the leg of the user and removed again without being damaged. To the upper part 10 there is fastened the resistance device 40, which bears against the upper part 20 and against the lower part 10 and provides an adjustable resistance to pivoting about the pivot axis 15. The sensors and the control device described above in connection with the exemplary embodiment of the prosthesis are correspondingly present also on the orthosis.

1. A method for controlling a prosthesis or orthosis of the lower extremity, having an upper part (10) and having a lower part (20) which is connected to the upper part (20) via a knee joint (1) and is mounted so as to be pivotable relative to the upper part (10) about a joint axis (15), wherein there is arranged between the upper part (10) and the lower part (20) an adjustable resistance device (40) by means of which, during walking, a flexion resistance (Rf) is changed on the basis of sensor data in an early and mid stance phase after initial heel contact up to the mid stance phase, characterized in that, after the initial heel contact, the flexion resistance (Rf) is increased to a value at which further flexion is blocked or at least slowed, wherein the temporal profile of the flexion resistance increase and/or the maximum achievable flexion angle (Af) is changed in dependence on the inclination of the surface or a height difference (ΔH) to be overcome.

2. The method as claimed in claim 1, characterized in that the maximum achievable flexion angle (Af) and/or the flexion angle (Af) at which the maximum flexion resistance (Rf) is achieved is increased in the case of an increasingly steep surface.

3. The method as claimed in claim 1, characterized in that, in the case of an increasingly steep surface, the maximum flexion resistance (Rf) is reduced.

4. The method as claimed in claim 1, characterized in that the flexion block or the flexion resistance increase is maintained for a defined period of time, and then the flexion resistance (Rf) is reduced.

5. The method as claimed in claim 4, characterized in that the flexion resistance is reduced after the flexion block or after the flexion resistance increase if a measure of the transverse force in the lower part (20) exceeds a limit value dependent on the inclination of the surface and/or a leg cord (70) exceeds a forward inclination dependent on the inclination of the surface and/or a measure of the hip moment initially exceeds and then falls below a limit value.

6. The method as claimed in claim 5, characterized in that the measure of the transverse force is determined by means of a transverse force sensor or from a difference in transverse force components of an ankle moment and knee moment.

7. The method as claimed in claim 1, characterized in that the flexion resistance (Rf) is reduced after an increase to below a blocking level if a predefined flexion angle (Af) is exceeded.

8. The method as claimed in claim 1, characterized in that the inclination of the surface can be calculated from a vertical and/or horizontal distance travelled in the preceding swing phase by the knee joint (1), in particular by a reference point in the vicinity of the sole of the foot, or from the ratio of a vertical and horizontal distance travelled in the preceding swing phase by the knee joint (1), in particular by a reference point in the vicinity of the sole of the foot, as a displacement calculation criterion.

9. The method as claimed in claim 8, characterized in that the beginning of the stance phase to be controlled is determined on the basis of an axial force impulse, a plantar flexion acceleration and/or an ankle moment.

10. The method as claimed in claim 1, characterized in that the inclination of the surface is calculated from an evaluation of a flexion angle (Af) and of an absolute angle of the upper part (10) or of the lower part (20) or from the evaluation of two absolute angles of the upper part (10) and lower part (20), as a kinematic criterion.

11. The method as claimed in claim 10, characterized in that the knee angular velocity and lower part angular velocity during walking are determined, and the quotient of the two angular velocities is calculated therefrom, wherein the inclination of the surface is determined on the basis of the change of the quotient of the angular velocities.

12. The method as claimed in claim 8, characterized in that the displacement calculation criterion and the kinematic criterion are used for determining the surface inclination.

13. The method as claimed in claim 1, characterized in that the position and/or orientation of a ground reaction force vector in relation to the orthosis or prosthesis is used as a control parameter.

14. The method as claimed in claim 1, characterized in that the detection of a roll-over of a foot part (30) over an edge prevents a flexion resistance increase or reduces the increased flexion resistance (Rf) again.
